(19) United States
(12) Patent Application Publication
(10) Pub. No.: US 2026/0196295 A1
(43) Pub. Date: Jul. 9, 2026

(54) METHOD AND APPARATUS FOR ANALYZING EXOSOMAL PROTEIN VARIANTS

(71) Applicants: KOREA UNIVERSITY RESEARCH AND BUSINESS FOUNDATION, Seoul (KR); EXOPERT CORPORATION, Seoul (KR)

(72) Inventors: Yeonho CHOI, Seoul (KR); Hyun Koo KIM, Seoul (KR); Seungmin KIM, Seoul (KR); Byeong Hyeon CHOI, Seoul (KR); Hyunku SHIN, Seoul (KR)

(73) Assignees: KOREA UNIVERSITY RESEARCH AND BUSINESS FOUNDATION, Seoul (KR); EXOPERT CORPORATION, Seoul (KR)

(21) Appl. No.: 19/133,392

(22) PCT Filed: Jan. 25, 2024
(86) PCT No.: PCT/KR2024/001171

§ 371 Date: May 28, 2025

(30) Foreign Application Priority Data
Jan. 25, 2023 (KR) 10-2023-0009725

(51) Int. Cl. G16B 20/20; G01N 21/65; G16B 40/10; G16B 40/20

(52) U.S. Cl. CPC G16B 20/20; G01N 21/658; G16B 40/10; G16B 40/20

(57) ABSTRACT

The present invention relates to a method and a device that can classify surface-enhanced Raman scattering (SERS) signals of protein contained in an exosome, which are abundant in the blood and stably transport a biomarker, through a convolutional neural network (CNN)-based artificial intelligence algorithm, and detect and monitor exosomal protein variants by mathematically interpreting the derived classification results. Through the present invention, it is possible to overcome the limitations of signal heterogeneity in existing technologies, thereby enabling simple and rapid non-invasive liquid biopsy of an exosomal protein in plasma.

### TECHNICAL FIELD

This application claims priority to Korean Patent Application No. 10-2023-0009725 filed in the Korean Intellectual Property Office on 25 Jan. 2023, the disclosure of which is incorporated herein by reference.

The present invention was also made with the support of the Ministry of Science and ICT of the Republic of Korea, under Project No. 2021M3H4A4079630 within Project Identification No. 1711159574, which was conducted in the research project named “Development of lung cancer diagnosis technology based on high-sensitivity multi-biomarker detection using nanoparticle printing technology and pattern analysis through machine learning” in the research program titled “Nano-Material Technology Development” by Korea University, under the management of the National Research Foundation of Korea, from 1 Feb. 2022 to 31 Jan. 2023.

The present invention was made with the support of the Ministry of Health and Welfare of the Republic of Korea, under Project No. HR14C0007060022 within Project Identification No. 1465036580, which was conducted in the research program named “Real-time Early Diagnostic Biochip for Exosome-based Cancer Detection” in the research project titled “Research-Driven Hospital fostering R&D” by Korea University ANAM Hospital under management of the Korea Health Industry Development Institute, from 1 Jan. 2022 to 10 Jan. 2023.

The present invention relates to a liquid biopsy technique for diagnosing and monitoring mutations by analyzing spectroscopic signals of exosomal proteins through artificial intelligence and, more specifically, to a method and a device capable of detecting and monitoring exosomal protein mutations by classifying surface-enhanced Raman scattering (SERS) signals of proteins contained in exosomes, which are abundant in the blood to stably transport biomarkers, through a convolutional neural network (CNN)-based artificial intelligence algorithm and mathematically analyzing the derived classification results.

### BACKGROUND ART

Exosomes, which are a type of extracellular vesicles that are abundantly found in body fluids, such as blood, urine, and saliva, are secreted by all cells and are involved in various biological mechanisms, such as intracellular communication and cancer metastasis. Exosomes, ranging in size from 30 to 200 nm, are formed when intraluminal vesicles are generated within cells via endocytosis and are released into the extracellular space by fusion of multivesicular endosomes with the plasma membrane via exocytosis. Therefore, exosomes contain substances, such as proteins and miRNAs, capable of representing the characteristics of parent cells, and can maintain stability even within fluids. Due to these properties, there has been a continuous endeavor to apply exosomes as biomarkers for liquid biopsy in the field of diagnostic medicines, but practical applications of exosomes are difficult due to high heterogeneity, which corresponds to an inherent property of almost all biologically derived substances.

Meanwhile, surface-enhanced Raman scattering (SERS) is a phenomenon in which Raman signals of molecules are amplified by a factor of at least 10⁷ to 10⁸, on the basis of the strong electromagnetic field generated in the nanogaps between plasmonic nanostructures. Through surface-enhanced Raman spectroscopy utilizing same, Raman signals of proteins can be obtained with ultra-high sensitivity, thereby effectively detecting subtle and localized structural such changes, as mutations. Especially, SERS exhibits high signal sensitivity even at low sample concentrations and is therefore significantly appropriate for the incorporation into mutation diagnosis technology through non-invasive liquid biopsy. However, the SERS signals of exosome protein have a limitation that complex and heterogeneous signals are detected due to intricate structures of proteins, interactions with plasmonic nanoparticles as signal detection probes, and the antibody distribution on the adherent surface.

To solve such a limitation, principal component analysis (PCA), partial least squares discriminant analysis (PLS-DA), or the like has been applied, but the analysis through such a technique as PCA or PLS-DA is not easy since exosomes present in patient body fluids are in a mixed state of exosomes derived from wild-type cells and exosomes derived from mutant cells. Moreover, mutant cell-derived are present at relatively low concentrations in the body, which hinders the development of diagnostic technologies for analytical samples containing a mixture of wild-type proteins of high concentrations and mutated proteins of low concentrations.

Hence, there is a need for the development of non-invasive biopsy techniques capable of overcoming the heterogeneity of SERS signals obtained from a specimen containing a mixture with mutated exosomal proteins of low concentrations.

### DISCLOSURE OF INVENTION

### Technical Problem

The present inventors verified that the identification the presence or absence of mutated proteins in a specimen and the classification of the mutation order of mutated proteins can be effectively achieved by constructing an artificial intelligence model to analyze SERS signals from exosomal proteins derived from a cell culture with a mixture of wild-type cells and mutated cells.

Accordingly, an aspect of the present invention is to provide a method for analyzing mutations of exosomal proteins in a specimen.

Another aspect of the present invention is to provide a computer program for analyzing mutations of exosomal proteins in a specimen.

Still another aspect of the present invention is to provide a computing device for analyzing mutations of exosomal proteins in a specimen.

### Solution to Problem

The present invention is directed to a method and a device for diagnosing and monitoring mutated proteins by analyzing SERS signals of exosomal proteins through artificial intelligence, and the method according to an embodiment of the present invention can identify the presence or absence of mutated proteins in a specimen and classify the mutation order of mutated proteins promptly and accurately.

Hereinafter, the present invention will be described in more detail.

In accordance with an aspect of the present invention, a method for analyzing mutations of exosomal proteins in a specimen is provided, the method being executed by a computing device, the method including: a generation step of generating input data based on surface-enhanced Raman scattering (SERS) signal data of the exosomal proteins in the specimen; and a detection step of determining the mutation status of the exosomal proteins on the basis of the input data, by using a pre-trained detection model.

As used herein, the term “specimen” refers to a sample containing exosomal proteins to be analyzed. The specimen may be obtained from a subject, and may include: intravascular fluids, such as blood, plasma, serum, and lymph; liquids, for example, mucosal secretions or glandular excretions, such as urine, pus, sputum, sweat, saliva, tears, and nasal discharge; and furthermore, mixtures of solid substances, such as feces, lesions, and biopsy tissues, and arbitrary liquids, but is not limited thereto.

As used herein, the term “subject” refers to a target on which exosomal protein mutations analysis is conducted by the analysis method according to an embodiment of the present invention. The subject may be a cell, a cell population, a tissue, an organ, or a living organism, where exosome secretion occurs, and specifically, the subject may be a mammal, such as a primate including a human, but is not limited thereto.

As used herein, the term “exosomal protein” refers to a protein contained in a specimen, and may collectively refer to a membrane protein connected to a phospholipid membrane of an exosome, or a protein contained in the exosomal lumen, or may refer to one or multiple target proteins selected from a series of selection processes among total proteins contained in the exosome.

As used herein, the term “mutation” refers to a trait that is expressed differently from the wild type, accounting for the majority of phenotypes within one species, wherein such a mutation results in significant differences in the phenotypic characteristics or behavioral patterns of the mutated individual compared with those of the wild-type individual. Especially, the individual, like the subject, may refer to a cell, a cell population, a tissue, an organ, or a living organism, where exosome secretion occurs, and specifically, the individual may be a mammal, such as a primate including a human. Especially, a trait expressed in an individual may involve the expression of a protein according to the gene.

As used herein, the term “input data” refers to the data that is first fed into a neural network model according to an embodiment of the present invention. The input data may be generated by performing a series of computations on SERS signal data electronically transformed from SERS signals measured from exosomal proteins in a specimen. The format of the input data is not particularly limited, and for example, may be tensor data.

As used herein, the term “tensor data” refers to a format of data composed of a multi-dimensional array of one or more values. The format of input data to a neural network model according to an embodiment of the present invention may be zero-, one-, two-, or higher-dimensional tensor data, and for example, may be one-dimensional vector data, but is not limited thereto.

As used herein, the term “training” refers to a process of optimizing each operation according to an algorithm constituting a neural network so that, with respect to the outcomes and decisions intended to be derived by a neural network, the neural network can derive the optimal outcomes or make optimal decisions. The neural network model according to an embodiment of the present invention may be trained using at least one manner of: supervised learning using training data labeled with ground truth; unsupervised learning using training data not labeled with ground truth; semi-supervised learning using both training data labeled with ground truth and training data not labeled with ground truth; or reinforcement learning for allowing model's actions according to the status to be optimized through rewards, and for example, the neural network model may be trained through supervised learning.

In an embodiment of the present invention, the method for analyzing mutations of exosomal proteins in a specimen may include a signal measurement step of measuring SERS signals from the exosomal protein in the specimen.

In an embodiment of the present invention, the generation step may include a statistical processing step of transforming multiple SERS signal data points into the mean signal through statistical processing.

As used herein, the term “mean signal” refers to a signal derived by conducting multiple SERS signal measurements on the exosomal proteins in the specimen and then statistically processing multiple SERS signals or multiple SERS signal data points, wherein the statistical processing may obtain the mean or the standard deviation of the signal data. Especially, the multiple SERS signal measurements may be conducted in a sufficient number to extract the overall features of the specimen. For example, the mean signal may be derived from 80 to 100 SERS signal measurements, but is not limited thereto.

In an embodiment of the present invention, the statistical processing step may be transforming 80 to 100 SERS signal data points into the mean signal through statistical processing.

In an embodiment of the present invention, the detection step may include: a production step of producing output information on the mutation status of the exosomal proteins by using a pre-trained detection model; and a similarity determination step of determining the relative similarity of the exosomal proteins in the specimen with wild-type and mutated exosomal proteins, on the basis of the output information.

As used herein, the term “output information” refers to the data outputted, according to a series of computations, from input data fed into a neural network model according to an embodiment of the present invention. The format of the output information may be the same as or different from the input data and, for example, the format of the output information may be zero-dimensional scalar data or one-dimensional vector data, but is not limited thereto.

As used herein, the term “relative similarity” refers to a criterion for determining which of wild-type exosomal proteins or mutated exosomal proteins the exosomal proteins in the specimen are more similar to, wherein the relative similarity may be determined by comparing data on the exosomal proteins in the specimen with data on the wild-type and mutated exosomal proteins to derive individual similarities, respectively, and comparing the derived individual similarities.

Especially, the derivation of individual similarities may employ any technique for quantifying similarity between the data and, for example, the individual similarity may be derived through the calculation of Mahalanobis distance between data on the exosomal proteins in the specimen and data on the wild-type or mutated proteins.

In an embodiment of the present invention, the output information may be one-dimensional tensor data with a length of 128.

In an embodiment of the present invention, the detection model may be trained through supervised learning on the basis of: training data containing SERS signal data generated from wild-type exosomal proteins and SERS signal data generated from mutated exosomal proteins; and detection labels with respect to the mutation status of the exosomal proteins corresponding to the training data, and the supervised learning may be performed using the detection model, on the basis of: training information generated from the training data by using the detection model; and the detection labels.

As used herein, the term “detection label” refers to binary data labeled as ground truth in the training data containing SERS signal data generated from wild-type exosomal proteins and SERS signal data generated from for the training of the mutated exosomal proteins, detection model according to an embodiment of the present invention. For instance, the detection model according to an embodiment of the present invention may be trained through supervised learning using detection labels assigned a value of 1 for a wild-type protein and a value of 0 for a mutated protein and training data.

In an embodiment of the present invention, the method may include a classification step of, when the exosomal proteins are determined as being mutated in the detection step, classifying the mutation order of the mutated exosomal proteins on the basis of the input data by using a pre-trained classification model.

As used herein, the term “mutation order” refers to classifying the number of mutation events in an individual with mutation and, for example, the mutation order classification in an individual may be achieved by classifying the first mutation as a primary mutation and any subsequent mutations as secondary or higher mutations. In the determination of secondary or higher mutations, both subsequent mutations occurring at different amino acid positions of the same protein as harboring the primary mutation and subsequent mutations occurring in a protein distinct from the protein harboring the primary mutation may be referred to as secondary or higher mutations.

In an embodiment of the present invention, when the exosomal proteins are determined as being mutated in the detection step, the classification step may be performed on exosomal proteins in a specimen that is newly obtained from the same subject after the lapse of a predetermined period following the termination of the detection step.

In an embodiment of the present invention, the classification step may include a mutation information generation step of generating mutation information by calculating the proportion of SERS signal data points determined as showing secondary or higher mutations among the total SERS signal data points.

As used herein, the term “total SERS signal data points” refers to a collection of multiple of SERS signal data generated on the basis of multiple SERS signals measured from the exosomal proteins in the specimen, and for deriving the mean signal, the multiple SERS signal data points generated from the exosomal proteins may also correspond to the total SERS signal data points. As used herein, the terms “total SERS signal data points” and “SERS signal data set” may be used interchangeably with each other.

As used herein, the term “mutation information” refers to the proportion of SERS signal data points determined as showing secondary or higher mutations among the total SERS signal data points obtained from the exosomal proteins in from the specimen a subject determined as having mutated exosomal proteins.

Especially, a predetermined cut-off value is applied to the proportion in the mutation information, so that a subject may be classified as having secondary or higher mutations if the proportion of mutation information is higher than the corresponding cut-off value.

In an embodiment of the present invention, the total SERS signal data points may be generated based on 100 SERS signals measured from the exosomal proteins in the specimen.

In an embodiment of the present invention, the classification model may be trained through supervised learning on the basis of: order training data containing SERS signal data according to the mutation order of the exosomal proteins; and classification labels with respect to the mutation of the exosomal proteins corresponding to the order training data, and the supervised learning may be performed using the classification model, on the basis of: training information generated from the mutation order training data by using the classification model; and the classification labels.

As used herein, the term “classification label” refers to scalar data labeled as ground truth in the order training data containing SERS signal data generated from exosomal proteins with primary mutation and SERS signal data generated from exosomal proteins with secondary or higher mutations, for the training of the classification model according to an embodiment of the present invention.

The number of classification labels may be set to any number to optimize the training of the classification model and, for example, 0 may be assigned to primary mutation, and 1 may be assigned to secondary mutation.

When three or more classification labels are set to train both primary mutation and secondary or higher mutations, a neural network needs to solve a multi-class classification issue. Since proteins with tertiary or higher mutations are present at significantly lower concentrations compared with wild-type proteins or proteins with primary and secondary mutations, the neural network model may exhibit degraded efficiency and performance during multi-class classification training.

Therefore, considering the training efficiency and performance of the mutation classification model, binary labels instead of multiple labels are assigned by stages even for the classification of tertiary or higher mutations, thereby effectively classifying higher-order mutations.

In another aspect of the present invention, a computer program stored in a storage medium is provided, wherein, when the computer program is executed on at least one processors, the computer program performs operations for analyzing mutations of exosomal proteins in a specimen, the operations including: an operation of generating input data based on SERS signal data of exosomal proteins in a specimen; and an operation of determining the mutation status of the exosomal proteins on the basis the input data by using a pre-trained detection model.

In still another aspect of the present invention, a computing device for analyzing mutations of exosomal proteins in a specimen is provided, the computing device including: a processor including at least one core; and a memory, wherein the processor generates input data based on SERS signal data of exosomal proteins in a specimen and determines the mutation status of the exosomal proteins on the basis the input data by using a pre-trained detection model.

### Advantageous Effects of Invention

The method and device for analyzing mutations of exosomal proteins according to the present invention can overcome the limitations of signal heterogeneity that are inherent in existing techniques, with respect to SERS signal analysis, and thus enable convenient and prompt non-invasive liquid biopsy on exosomal proteins in the plasm. Accordingly, the present invention is expected to replace conventional invasive and non-invasive biopsy techniques for disease diagnosis.

### BRIEF DESCRIPTION OF DRAWINGS

FIG. 1 is a block diagram of a computing device for implementing operations for analyzing mutations of exosomal proteins in a specimen according to an embodiment of the present invention.

FIG. 2 is a schematic diagram showing the structure of a neural network model constituting a detection model or classification model according to an embodiment of the present invention.

FIG. 3 is a flow chart illustrating a procedure for analyzing mutations of exosomal proteins in a specimen according to an embodiment of the present invention.

FIG. 4 is a schematic diagram showing a procedure for analyzing mutations of exosomal proteins in a specimen according to an embodiment of the present invention.

FIG. 5 presents graphs showing SERS signal trends obtained from a wild-type exosomal protein and a lung cancer cell-derived exosomal protein.

FIG. 6 is a schematic diagram showing a procedure of acquiring SERS signal data sets from substrates with immobilized wild-type cell- and mutated cell-derived exosomal proteins.

FIG. 7 presents graphs showing accuracy and loss of training of a detection model according to an embodiment of the present invention.

FIG. 8 presents graphs showing accuracy and loss of training of a classification model according to an embodiment of the present invention.

FIG. 9 is a schematic diagram showing a procedure of determining the relative similarity of exosomal proteins in a subject with wild-type and mutated exosomal proteins to detect the mutation status, through a detection model, according to an embodiment of the present invention.

FIG. 10 presents graphs showing the results of classifying normal persons and lung cancer patients on the basis of the relative similarity of exosomal protein SERS signals, determined through a detection model according to an embodiment of the present invention.

FIG. 11 is a ROC graph showing the mutated exosomal protein detecting performance of the detection model according to an embodiment of the present invention.

FIG. 12 is a schematic diagram showing a procedure of classifying changed mutation orders occurring according to the long-term treatment of a patient through the classification model according to an embodiment of the present invention.

FIG. 13 is a graph showing the results of classifying lung cancer patients into patients with primary mutation and patients with secondary mutation on the basis of mutation information generated through the classification model according to an embodiment of the present invention.

FIG. 14 shows the proportions of signals determined as corresponding to secondary mutation among 100 exosomal protein SERS signals obtained at the time of primary mutation diagnosis (t₁) and the time of secondary mutation diagnosis (t₂), respectively, in subjects classified as patients with secondary mutation through the detection model according to an embodiment of the present invention.

FIG. 15 shows the rates of increase/decrease in secondary mutation signals at the time of primary mutation diagnosis (t₁) and the time of secondary mutation diagnosis (t₂) in subjects classified as patients with secondary mutation through the detection model according to an embodiment of the present invention.

### BEST MODE FOR CARRYING OUT THE INVENTION

The present invention is directed to a method for analyzing mutations of exosomal proteins in a specimen, the method being executed by a computing device, the method including:
a generation step of generating input data based on
surface-enhanced Raman scattering (SERS) signal data of the exosomal proteins in the specimen; and
a detection step of determining the mutation status of the exosomal proteins on the basis of the input data, by using a pre-trained detection model.

### MODE FOR CARRYING OUT THE INVENTION

Hereinafter, preferable embodiments of the present invention will be described with: reference to the accompanying drawings. A detailed description to be disclosed below together with the accompanying drawings is to describe the exemplary embodiments of the present invention and does not represent the sole embodiment for carrying out the present invention, and these exemplary embodiments are not construed to limit the scope of the present invention.

In some cases, known structures and devices may be omitted or block diagrams mainly illustrating key functions of the structures and devices may be provided so as to not obscure the concept of the present invention. Throughout the specification, like reference numerals will be used to refer to like elements.

Throughout the specification, when a certain portion “comprises”, “contains”, or “includes” a certain component, this means that the certain portion may further comprise, contain, or include other components, rather than excluding the other components, unless otherwise specifically stated.

Throughout this specification and the claims that follow, when it is described that an element is “connected” to another element, the element may be “directly connected” to the another element or “connected” to the another element with a third element disposed therebetween.

The term “or” is intended to mean an inclusive “or” rather than an exclusive “or.” That is, when the combination or use among components is not otherwise specified or clear from the context, the phrase “X employs A or B” may be applied to encompass any of the following instances: “X employs A”, “X employs B”, or “X employs both A and B”, and the phrase “X is connected to A or B” may be applied to encompass any of the following instances: “X is connected to A”, “X is connected to B”, or “X is connected to both A and B”.

The term “ . . . unit” used herein refers to a unit that performs at least one function or operation and may be implemented in hardware, software, or a combination thereof. Furthermore, “a”, “one”, and the like may be used to include both the singular form and the plural form unless indicated otherwise in the context of the present invention or clearly denied in the context.

It shall be appreciated that the various illustrative logical blocks, configurations, modules, circuits, means, logics, and algorithm steps described in connection with the embodiments disclosed herein may be implemented as electronic hardware, computer software, or combinations thereof. To clearly illustrate the hardware and software, various interchangeability of illustrative components, blocks, configurations, means, logics, modules, circuits, and steps have been described above generally in terms of their functionality. Whether the functionality is implemented as hardware or software depends on a specific application and design constraints imposed on the overall system. Those skilled in the art may implement the described functionality in various ways for each of specific applications. However, decisions of such implementations shall be interpreted without departing from the scope of the present disclosure.

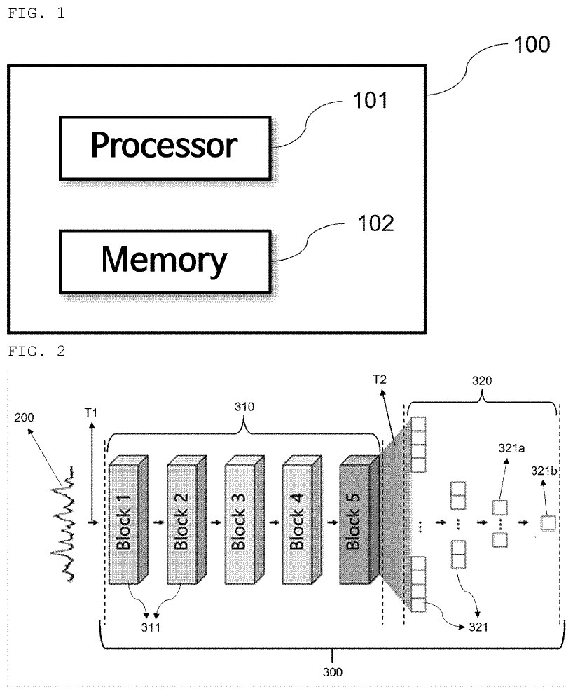

FIG. 1 is a block diagram of a computing device performing operations for analyzing mutations of exosomal proteins in a specimen according to an embodiment of the present invention.

Referring to FIG. 1, a computing device 100 performing operations for analyzing mutations of exosomal proteins in a specimen according to an embodiment of the present invention may include a processor 101 and a memory 102.

The processor 101 may perform an operation of generating input data based on surface-enhanced Raman scattering (SERS) signal data of the exosomal proteins in the specimen.

SERS signal data may be generated by a series of pre-processing on SERS signals of the exosomal proteins in the specimen obtained from a subject. For example, SERS signals may be transformed into data suitable for neural network model training through standardization or min-max normalization.

The processor 101 may transform SERS signal data into input data in a tensor format. The input data may have any data format, for example, a one-dimensional vector format or a two-dimensional matrix format.

The processor 101 may perform an operation of transforming multiple SERS signal data points into the mean signal through statistical processing. The multiple SERS signal data points may be generated by pre-processing SERS signals obtained by multiple measurements on a single specimen, and multiple SERS signal data points generated from a single specimen may constitute a SERS signal data set. The statistical processing technique for transforming the multiple SERS signal data points into the mean signal is not limited to a particular method. For example, the mean and standard deviation of each component of multiple SERS signal data points are derived and transformed into the mean signal.

The processor 101 may perform an operation of determining the mutation status of exosomal proteins on the basis the input data by using a pre-trained detection model. The pre-trained detection model may be a neural network model and, particularly, a deep neural network model.

The deep neural network is an artificial neural network where several hidden layers are present between an input layer and an output layer, with one or more nodes constituting each layer and one or more edges connecting the nodes. The deep neural network can model complex non-linear relationships through computations from the input layer to the output layer. Such a neural network may be referred to as “network” or “neural network”, which are used exchangeably with each other, and such a deep neural network may be referred to as “deep neural network”, which are used exchangeably with each other.

Deep neural networks may be classified, according to the algorithm of the computation from the input layer to the output layer, into a convolutional neural network (CNN), a recurrent neural network (RNN), an auto encoder, a generative adversarial network, a restricted Boltzmann machine (RBM), a deep belief network (DBN), a Q-network, a U-network, or a Siamese network, but are not limited thereto.

When the detection model is a deep neural network model, the structure of the detection model may be composed of a combination of any deep neural network models. For example, the detection model may be composed of a combination of a convolutional neural network including multiple layers of convolutional unit blocks and a fully-connected neural network including multiple layers of fully-connected layers, but is not limited thereto.

The convolutional neural network is a type of deep neural network model, which is inspired by the visual cortex of animals and designed for processing data having multi-dimensional patterns, such as images and sounds. The convolutional neural network may generally include a convolution layer, a pooling layer, and a fully-connected layer. One or more convolutional layers and pooling layers may be repeatedly present in the convolutional neural network, and the input values fed into the convolutional neural network may be transformed into output values through such a layered structure. AlexNet, VGGNet, ResNet, and the like are representative structures of the convolutional neural network, but the structure of the convolutional neural network according to an embodiment is not limited thereto.

The fully-connected neural network may include an input layer, an output layer, and several hidden layers present therebetween. In the fully connected neural network, when one or more nodes of each adjacent layer form an input node and output node relationship between the adjacent layers, all nodes of a layer composed of input nodes may be connected to all nodes of a layer composed of output nodes through edges, respectively.

Especially, the node may refer to an object representing one data unit, and in a relationship between an input node and an output node connected by an edge, the data of the output node may have a value, which is determined on the basis of the data fed into the input node. The node may be referred to as “neuron”, and may be used interchangeably therewith.

The edge may have a weight, and may further have a bias. The weight or the bias may be variable, and the edge may determine a value of the output node by performing computations reflecting the weight or bias on a value fed into the input node. The edge may be referred to as “link”, and may be used interchangeably therewith.

The processor 101 may perform an operation of producing output information for the mutation status of the exosomal proteins by using the pre-trained detection model. Specifically, when the input data generated from SERS signals of the exosomal proteins in the specimen are fed into the detection model, output information for the mutation status of the exosomal proteins in the specimen may be produced as a final output, and the data format of the output information may be zero-, one-, two-, three-, or higher-dimensional tensor format, and for example, the output information may be in the format of zero-dimensional scalar data or one-dimensional vector data.

The processor 101 may perform an operation of determining the relative similarity of the exosomal proteins in the specimen with wild-type and mutated exosomal proteins, on the basis of the output information. In the production of the output information for determining relative similarity, the output information may be produced in the format of a scalar having a single component or a vector having two or more components. Especially, the exosomal proteins in the specimen is in a mixed state of mutated proteins and wild-type or normal proteins, and thus the mutation status cannot be determined through simple binary classification. Therefore, the output information can be produced in a vector format when the relative similarity for determining the mutation status is determined.

When the Mahalanobis distance between data on the exosomal proteins in the specimen and data on wild-type or mutated proteins is calculated for the determination of relative similarity, the Mahalanobis distance may be calculated by deriving the mean and covariance of individual components of the output information outputted in a vector format for each data point and then reflecting the covariance on the difference between the means of each output information.

When the exosomal proteins are determined as being mutated in the detection step, the processor 101 may perform an operation of classifying the mutation order of the mutated exosome proteins on the basis of the input data by using a pre-trained classification model. The pre-trained classification model may be a neural network model, particularly, a deep neural network model.

When the classification model is a deep neural network model, the structure of the classification model may be composed of a combination of any deep neural network models. For example, the classification model may be composed of a combination of a convolutional neural network including multiple layers of convolutional unit blocks and a fully-connected neural network including multiple layers of fully-connected layers, but is not limited thereto.

The process 101 may perform an operation of generating mutation information by calculating the proportion of SERS signal data points determined as showing secondary or higher mutations among the total SERS signal data points. The mutation information may be generated on the basis of input data fed into the classification model, or input data separately generated.

The process 101 may produce output data for determining secondary or higher mutations from the input data when calculating the proportion of SERS signal data points determined as showing secondary or higher mutations among the total SERS signal data points. The output data for determining secondary or higher mutations may be in the format of zero-dimensional scalar data, one-dimensional vector data, two-dimensional matrix data, or three- or higher-dimensional forms. For example, the output data may have scalar values for binary classification of the presence or absence of secondary or higher mutations for particular input data.

The processor 101 may perform calculations for the training of a neural network, such as generation of input data for training the neural network model, error calculation, and weight optimization for computations of the neural network model using backpropagation.

The processor 101 may be composed of at least one core, and may include a processor for data analysis and deep learning, such as a central processing unit (CPU), a graphics processing unit (GPU), or a tensor processing unit (TPU) in the computing device.

The processor 101 may read a computer program stored in the memory 102 to perform data processing for the training of the neural network model according to an embodiment of the present invention, and may perform computations for the training of the neural network model.

The memory 102 may store any format of information generated or determined by the processor 101, and may include at least one type of storage medium among a flash memory type-, hard disk type-, multimedia card micro type-, and card-type memories (e.g., SD or XD memory), random access memory (RAM), static random access memory (SRAM), read-only memory (ROM), electrically erasable programmable read-only memory (EEPROM), programmable read-only memory (PROM), magnetic memory, magnetic disk, and optical disk.

The computing device 100 for performing operations for analyzing mutations of exosomal proteins in a specimen according to an embodiment may further include, as necessary: a network unit for transmitting and receiving information containing SERS signals of the exosomal proteins in the specimen or other data; and a database for storing and managing data used in a computing procedure.

Therefore, the computing device 100 for performing operations for analyzing mutations of exosomal proteins in a specimen according to an embodiment of the present invention enables wired or wireless information exchange with other computing devices, terminals, or electronic equipment, capable of measuring SERS signals of the exosomal proteins in the specimen. Furthermore, the computing device according to an embodiment of the present invention enables wired or wireless information exchange with other computing devices, terminals, or storage memories, or cloud servers, each storing SERS signal data of wild-type exosome proteins or SERS signal data of mutated exosomal proteins.

In addition, the data generated in the performance of operations for analyzing mutations of exosomal proteins in a specimen, for example, SERS data of exosomal proteins and input data generated therefrom, weight data of the detection model and the classification model, output information produced from input data, and relative similarity of exosomal proteins in a specimen with wild-type and mutated exosomal proteins, may be stored in the database of the computing device 100 according to an embodiment of the present invention, and the data stored in the database may be managed, such as by deletion or modification, by an external input applied from a user.

FIG. 2 is a schematic diagram showing the structure of a neural network model constituting a detection model or classification model according to an embodiment of the present invention.

Referring to FIG. 2, a detection model or classification model for analyzing mutations of exosomal proteins in a specimen according to an embodiment of the present invention may be a neural network model 300.

The neural network model 300 may be trained toward the minimization of errors in the output. For example, an error may be calculated by comparing the output of the neural network with guide labels of training data through supervised learning, and an error may be calculated by comparing the output of the neural network with training data through unsupervised learning. The calculated error may be backpropagated from an output layer to an input layer in the neural network, and the weight or bias of each edge connecting nodes of the neural network may be changed according to the backpropagation, and training may proceed until the weight and bias are determined to minimize the error of the output. The degree of change in weight or bias according to each training may be determined depending on the learning rate.

The neural network model 300 may be trained through supervised learning on the basis of training data, containing SERS signal data, and guide labels.

In a case where the neural network model 300 is a detection model, the training data may contain SERS signal data generated from wild-type exosomal proteins and SERS signal data generated from mutated exosomal proteins, and the guide labels may be detection labels labeled as ground truth, with 0 and 1, or other any numbers, with respect to the mutation status of exosomal proteins, corresponding to the training data.

In a case where the neural network model 300 is a classification model, the training data may be mutation order straining data containing SERS signal data according to the mutation order of exosomal proteins, and the guide labels may be classification labels labeled as ground truth, with 0 and 1, or other any numbers, with respect to the mutation order of exosomal proteins, corresponding to the order training data.

The training data may contain multiple SERS signal data points measured from multiple specimens containing wild-type exosomal proteins and mutated exosomal proteins, and the training data may contain a sufficient number of SERS signal data points for training of the neural network model 300. The training data used in the detection model and the training data used in the classification model contain SERS signal data measured from the same specimen, but each data point may be labeled differently.

When overfitting for training data occurs during the training of the neural network model 300, the accuracy of protein mutation analysis results for a specimen obtained from a subject may be poor. To avoid this issue, a technique, such as regularization, dropout, or batch normalization, may be applied to input data, and input values for each layer.

The input data fed into the neural network model 300 may be generated by a first transformation (T₁) performed on the SERS signal 200 of the exosomal proteins.

The SERS signals 200 may be measured from the exosomal proteins in a specimen obtained from a subject. The SERS signal measurement on the exosomal proteins may be conducted on a single specimen one time or multiple times, and thus one or multiple SERS signals 200 may be obtained from a single specimen. For example, 80 to 100 SERS signals 200 may be obtained for a single specimen, but are not limited thereto.

The SERS signal 200 may be transformed into input data via the first transformation (T₁). The first transformation (T₁) may be any method capable of transforming the SERS signal 200 into a format suitable to be fed into the neural network model 300. For example, the SERS signal 200 may be transformed into input data by digitization in the format of vector data followed by normalization using the min-max normalization technique during the first transformation (T₁).

The input data may be in the format of a one-dimensional vector having two or more components, for example, a vector having 800 components, but are not limited thereto.

The neural network model 300 may include a convolutional neural network 310 and a fully-connected neural network 320.

The convolutional neural network 310 may include one or more convolutional unit blocks 311, for example, five convolutional unit blocks 311.

The convolutional unit block 311 may include one or more convolutional layers, and may further include batch normalization, activation function, and pooling layers.

The convolutional layer may extract features from the input value by using a filter. The filter may be in the format of a two-dimensional matrix, and each component of the filter may correspond to a parameter that is optimized through training of the neural network model 300 according to an embodiment of the present invention. For example, when the filter is a 3×1 matrix, data features corresponding to the corresponding filter may be extracted and optimized during the training process of the neural network model 300.

The convolutional layer may include at least one filter, and when the convolutional layer extracts features from the input value through the at least one filter, the output value may have a depth equal to the number of filters used for feature extraction. For example, when an input value in the format of an 800×1 matrix is computed using four filters, the output value will have a depth of 4. Therefore, when input data in a vector format is fed into the convolutional neural network 310 according to an embodiment of the present invention, the output value obtained by a series of computations through the convolutional unit blocks 311 has a three-dimensional tensor data format.

The output value outputted by all the computations of the convolutional neural network 310 may be the first input value to the fully-connected neural network 320 through a second transformation (T₂).

The second transformation (T₂) may be any method capable of transforming the output value outputted from the convolutional neural network 310 into a format suitable to be fed into the fully-connected neural network 320. For example, an output value in the format of a two-dimensional matrix outputted from the convolutional neural network 310 may be transformed into the first input value fed into the fully-connected neural network 320 through flattening for allowing all the components of the matrix to be flattened into a one-dimensional array. Especially, batch normalization is performed on the first input value that has been flattened, thereby improving the training efficiency of the fully-connected neural network 320.

The fully-connected neural network 320 may include one or more fully-connected layers 321, for example, four fully-connected layers 321.

The fully-connected layer 321 may be a generic term of an input layer, hidden layers, and an output layer. The first input value fed into the fully-connected neural network 320 may have a gradually decreased size through each layer and, for example, a single scalar value may be ultimately outputted at the output layer.

The computations between layers, excluding the output layer, in the fully-connected layer 321 may include a computation according to an activation function, such as the sigmoid function, tanh function, ReLU function, ELU function, or Maxout function.

In an embodiment of the present invention, a hidden layer adjacent to the output layer in the fully-connected layer 321 may be referred to as a final vector output layer 321a, and the output layer in the fully-connected layer 321 may be referred to as a final binary output layer 321b.

In the detection or classification step with respect to the mutations of exosomal proteins in a specimen, the neural network model 300 may use an output value of the final vector output layer 321a and an output value of the final binary output layer 321b.

The output value of the final vector output layer 321a may be in the format of a vector having a length of at least 2, for example, vector data having a length of 128.

The output value of the final vector output layer 321a may be used in the determination of the mutation status of exosomal proteins through the detection model according to an embodiment of the present invention, and in such a situation, the output value of the final vector output layer 321a may be used interchangeably with “output information”, which is a basis to determine relative similarity.

The output value of the final binary output layer 321b may be scalar data having a single value, for example, scalar data having a value of 0 or 1.

The output value of the final binary output layer 321b may be used in the classification of the mutation order of proteins through the mutated exosomal classification model according to an embodiment of the present invention. For example, when the output value of the final output layer 321b is 0, the binary classification model may determine that secondary or higher mutations do not occur in the mutated exosomal proteins, and when the output value of the final binary output layer 321b is 1, the classification model may determine that secondary or higher mutations occur in the mutated exosomal proteins.

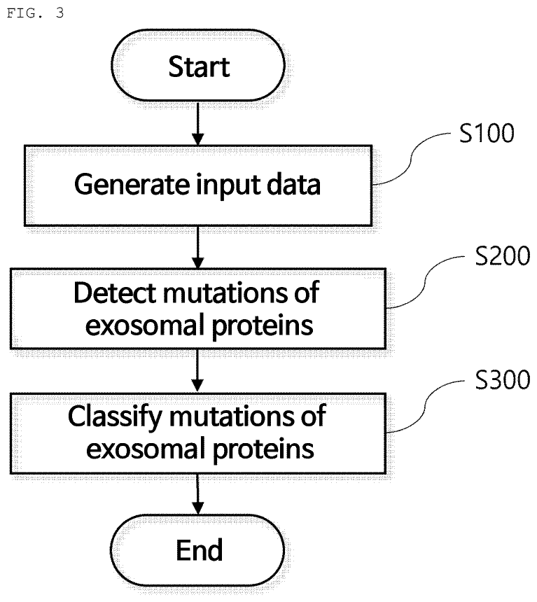

FIG. 3 is a flow chart illustrating a procedure for analyzing mutations of exosomal proteins in a specimen according to an embodiment of the present invention.

Referring to FIG. 3, the procedure of analyzing mutations of exosomal proteins in a subject according to an embodiment of the present invention may include an input data generation step S100, a mutated exosomal protein detection step S200, and a mutated exosomal protein classification step S300.

In the input data generation step S100, the computing device according to an embodiment of the present invention may generate input data based on SERS signal data of the exosomal proteins in the subject. The input data may be generated based on the mean signal obtained by statistical processing of multiple SERS signal data points.

In the input data, input data generated based on SERS signals measured from wild-type or mutated exosomal proteins but not exosomal proteins in the specimen obtained from the subject, may be used as data for training of the detection model and the classification model according to an embodiment of the present invention.

In the mutated exosomal protein detection step S200, the computing device may determine the mutation status of exosomal proteins on the basis of the input data, by using a pre-trained detection model. Especially, the computing device may produce output information with respect to the mutation status of exosomal proteins by using the pre-trained detection model and then determine the relative similarity of exosomal proteins in the specimen with wild-type and mutated exosomal proteins on the basis of the output information, thereby determining which of wild-type exosomal proteins and mutated exosomal proteins the exosomal proteins in the specimen are more similar to.

In the mutated exosomal protein classification step S300, the computing device may classify the mutation order of the mutated exosome proteins on the basis of the input data by using the pre-trained classification model when the exosomal proteins are determined as being mutated in the detection step. Especially, the computing device determines how many data points result from secondary or higher mutations among the total SERS signal data points of the exosomal proteins in the subject and calculates the proportion thereof, thereby generating mutation information based on the proportion. The computing device may compare the cut-off value, which is a criterion for determining secondary or higher mutations, the mutation information, thereby classifying the exosomal proteins in the corresponding specimen as having secondary or higher mutations.

The mutated exosomal protein classification step S300 may be performed on the basis of the same input data as used in the detection step S200, or may be performed on the basis of new input data generated from the SERS signals measured from exosomal proteins newly obtained from the same subject after the lapse of a predetermined period following the termination of the detection step S200 performed on the same subject. Therefore, the mutated exosomal protein classification step S300 enables the classification of the protein mutation order in a current subject as well as the monitoring of the protein mutation status over time in the subject.

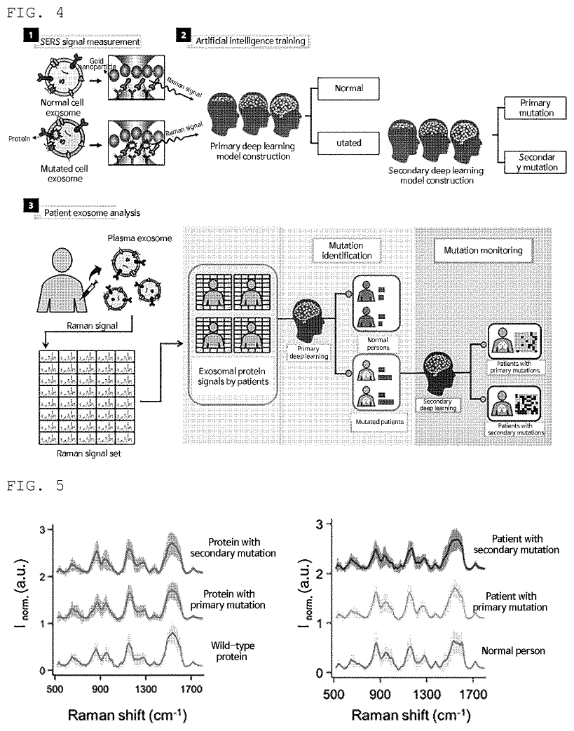

A schematic diagram of a procedure for analyzing mutations of exosomal proteins in a specimen according to the steps S100 to S300 is shown in FIG. 4.

A computer program for allowing the computing device 100 according to an embodiment of the present invention to perform operations including steps S100 to S300 to analyze the mutations of exosomal proteins in a specimen may be provided stored in a storage medium.

A computer program may be configured of a series of codes or algorithms, and according to the configuration of the computer program, the computer program may instruct, either independently or in combination, a program processing device, for example, a computing device (100), so as to enable the device to operate, or be interpreted by the processing device. To instruct the processing device or be interpreted by the processing device, the computer program may be embodied through any type of machine, component, physical device, computer, or storage medium. Furthermore, the computer program may be stored or executed in a distributed manner through a non-physical medium, such as a network-connected computer system or a cloud server.

The storage medium may continuously store the computer programs or temporarily store the computer programs for execution or downloading. The storage medium may be any one of various recording media or storage media in which a single piece or plurality of pieces of hardware are combined, and the medium is not limited to a medium directly connected to a computer system and may be distributed on a network. Examples of the storage medium include magnetic media, such as a hard disk, a floppy disk, and a magnetic tape, optical media, such as CD-ROM and DVD, magneto-optical media such as a floptical disk, and ROM, RAM, and a flash memory, which are configured to store computer programs.

Hereinafter, the present invention will be described in more detail by the following exemplary embodiments. However, these exemplary embodiments are used only for illustration, and the scope of the present invention is not limited by these exemplary embodiments.

### Example 1: Acquisition of SERS Signals of Wild-Type cell- and mutated cell-derived exosomal proteins

To detect SERS signals of exosomal proteins, a self-assembled monolayer (SAM) structure with 100-nm spherical gold nanoparticles regularly arranged was used.

Specifically, a cleaned cover glass was treated with APTES so as to induce a SAM structure on the surface thereof, and an antibody specifically binding to a target protein was attached, and BSA treatment was conducted to prevent unspecific binding. Thereafter, a sample containing lysed exosomes was incubated on the surface of the cover glass to induce the target protein to bind to the antibody. After concentrated gold nanoparticles were scattered on the target protein-immobilized cover glass, the nanoparticles were uniformly deposited on the cover glass by centrifugation, followed by drying. After the completion of the drying, the target protein-immobilized cover glass was measured for SERS signals. The results are shown in FIG. 5.

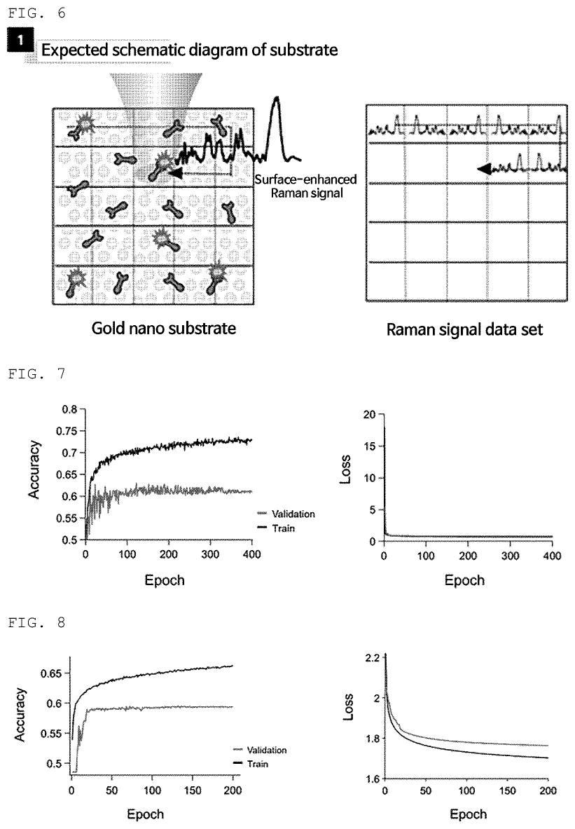

Especially, as for exosome samples derived from wild-type cells (A549), primary mutant cells (H838: L858R, HCC82: 19del), and secondary mutant cells (H1975: L858R+T790M, HCC827: 19del+T790M), 300 SERS signal data sets were acquired from each sample, and the signal data sets acquired from each sample were statistically analyzed to obtain the mean data and standard deviation data, thereby generating the mean signal for each sample. A schematic diagram of a signal data set acquiring process is shown in FIG. 6.

### Example 2: Artificial Intelligence Training Using Acquired Exosomal Protein SERS Signals

The CNN model extracts the relationship for spatially adjacent signals, and thus derives abstracted features of all the signals. The CNN model was fundamentally developed for analysis of two-dimensional images, but in the present invention, the CNN model was applied in the analysis of Raman signals, one-dimensional data, so that the CNN model was somewhat simplified and modified.

Furthermore, in order to construct a model capable of classifying signals of cell-derived wild-type exosomal proteins and signals of mutated exosomal proteins with high accuracy and low loss and favorably classifying signals of plasma proteins as well as cellular proteins, various parameters and structures were optimized.

Specifically, the exosomal protein SERS signals used in the training of the present model were pre-processed through min-max normalization, and Raman spectra in the range of 517-1800 cm⁻¹ were used as data.

The fully-connected layer (fc 128) at the end of the structure of the present model has a structure where 128 values are a single output, and on the basis of this structure, SERS signal data of wild-type exosomal proteins and mutated exosomal proteins were ultimately classified.

Especially, in order to overcome the most significant limitation in the use of bio-derived substances, such as exosomes in body fluids, for protein mutation diagnosis, that is, target proteins of low concentrations due to the mixing with wild-type proteins, a mean derivation value of several signals, instead of a single signal, was used as a final prediction derivation value assigned to each signal, so that in spite of the existence of target proteins at a low concentration in a sample, the overall features of the sample were extracted to determine the mutation status in the sample.

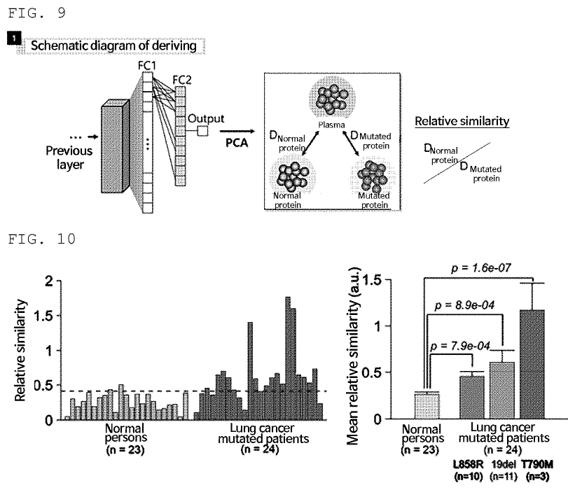

In the training of the present model, the trends of learning accuracy and loss over training iterations upon the training and validation of each of the detection model and the classification model were measured, and are shown in FIGS. 7 and 8.

As a result of training, both the detection model and the classification model showed an accuracy of 68-70% when a single SERS signal was used for training, but showed accuracies of at least 99% and at least 84%, respectively when 80 or more signal derivation values per sample were statistically analyzed. It was therefore identified that the use of a mean signal value in model training ensured higher accuracy.

### Example 3: Analysis of Exosomal Proteins Derived from Blood of Subjects

### 3-1. Determination of Mutation Status of Exosome Proteins

Since exosomal proteins derived from the blood of a mutated patient are mixed with wild-type proteins, it was hard to apply binary classification to the exosomal proteins, unlike the classification of cell-derived exosome proteins. The present invention used the information of the final vector output layer, which was a layer immediately before the final binary output layer, instead of the final binary output layer. Especially, the final vector output layer had 128 numerical values, which were derived through all the preceding layers. A schematic diagram showing a procedure of detecting mutated exosomal proteins is shown in FIG. 9.

Specifically, exosomal proteins in the blood of the subject were measured for SERS signals by the same method as in Example 1, and the input data generated based on the SERS signals were fed into a trained deep learning model to derive 128 values from the final vector output layer. Thereafter, the values derived from the final vector output layer in the subject were compared with the values from the final vector output layers in the wild-type cell- and mutated cell-derived exosomal proteins to analyze which of signals the signal in the subject are similar to.

Specifically, on the basis of the relative similarity criterion, the similarity of the final vector output layer of the subject with the final vector output layers of the wild-type cell- and mutated cell-derived exosomal proteins was analyzed. The relative similarity criterion was expressed as “the Mahalanobis distance with wild-type proteins/the Mahalanobis distance with mutated proteins”, wherein the Mahalanobis distance indicates dissimilarity between data points.

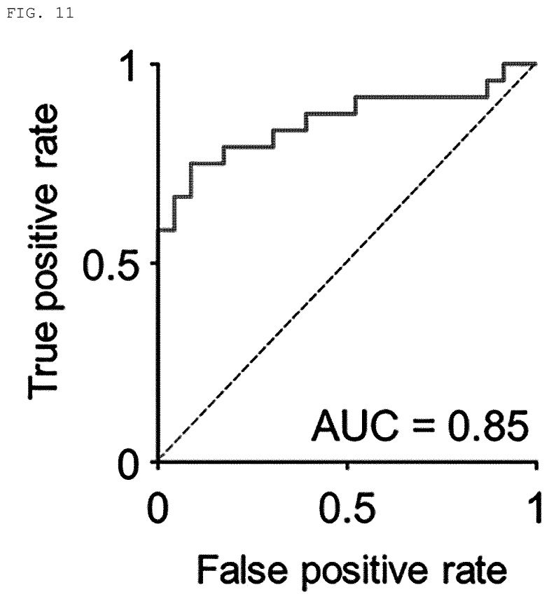

The results of classifying normal persons and lung cancer patients with mutations (L858R, 19del, T790M) on the basis of the relative similarity are shown in FIG. 10, and the mean relative similarity values between normal persons and lung cancer patients with mutations are shown in Table 1.

TABLE 1

| | Normal | | | |
|---|---|---|---|---|
| | persons | L858R | 19del | T790M |
| | (n = 23) | (n = 10) | (n = 11) | (n = 3) |
| Mean relative | 0.263 | 0.45 | 0.61 | 1.66 |
| similarity (a.u.) | | | | |

As a result of classification, statistically significant classifications of normal persons and lung cancer patients with mutations were confirmed with the naked eye, and significant classifications in various types of mutations were also confirmed with the naked eye.

Furthermore, in order to validate the diagnostic effect of the detection model trained according to the present invention, the ROC curve is shown in FIG. 11, and the AUC values, compared with data in existing documents, are shown in Table 2.

TABLE 2

| Author | Diagnostic method | AUC |
|---|---|---|
| Paolo Cotzia, et. al. | Plasma sequencing based | 0.63 |
| | ctDNA detection | |
| Lei Wang, et. al. | PCR and SERS based ctDNA | 0.85 |
| | detection | |
| Cheng-Yu Chen, et. al. | CT image-Radiomics | 0.89 |
| | based classification | |
| H. Alvarez, et. al. | dd-PCR based exoDNA | 0.66-0.85 |
| | mutation detection | |
| Present invention | Deep-learning and SERS | 0.85 |
| | based classification | |

Referring to FIG. 11 and Table 2, the detection model of the present invention showed an AUC value of 0.85, indicating usability after the diagnostic method using CT image-Radiomics (Cheng-Yu Chen, et al.), and the highest usability in terms of a liquid biopsy-based lung cancer diagnostic method. Consequently, the present invention is a highly accurate diagnostic method and is considered to be determined to have the potential to be applicable to actual diagnostics.

### 3-2. Classification of Mutation Order of Mutated Exosomal Proteins

Exosomal proteins derived from patients with primary mutations had a mixture of wild-type proteins and proteins with primary mutations, but exosomal proteins derived from patients with secondary mutations had a mixture of a large amount of wild-type proteins, proteins with primary mutations, and a tiny amount of proteins with secondary mutations. Even though mutations are classified through overall similarity values as in Example 3-1, classification is difficult to perform since the characteristics of the proteins with secondary mutations are not well reflected.

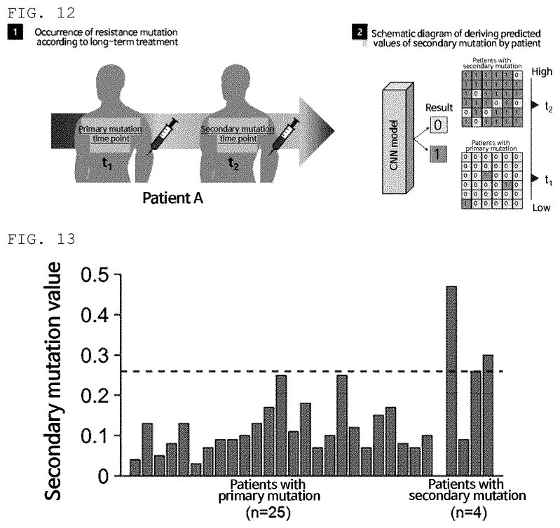

Therefore, the number of signals determined as secondary mutations among 100 signals per sample was calculated through the deep learning model, and the number of signals determined as secondary mutations was divided by the total number of signals (n=100), thereby deriving a secondary mutation value of exosomal proteins derived from a subject, that is, mutation information. A schematic diagram showing a procedure of classifying mutated exosomal proteins is shown in FIG. 12, and the results of classifying patients with primary mutation and patients with secondary mutation among lung cancer patients on the basis of the mutation value is shown in FIG. 13.

As a result of classification, it was confirmed with a naked eye that patients with primary mutation and patients with secondary mutation were well classified on the basis of the cut-off value for mutation classification.

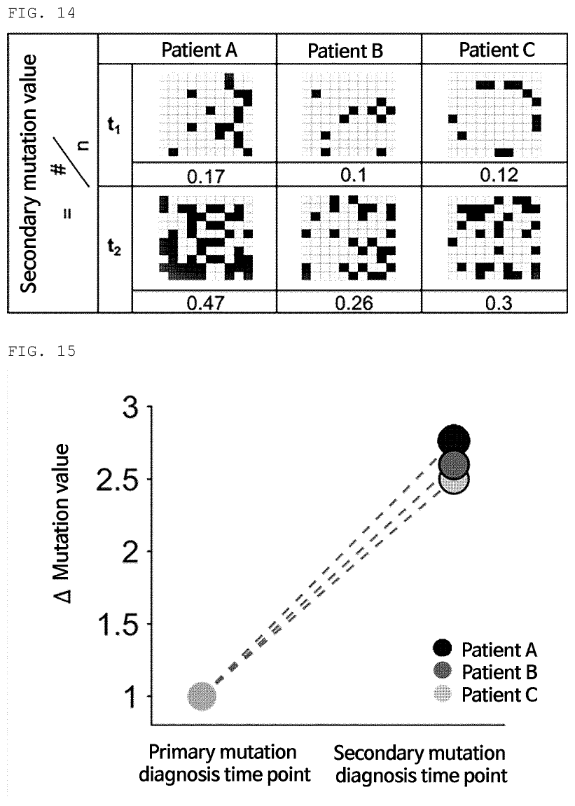

Additionally, the results of visualizing the predicted values classifying patients with primary mutation and patients with secondary mutation among lung cancer patients, on the basis of mutation information at the time of primary mutation diagnosis (t1) and the time of secondary mutation diagnosis (t2) are shown in FIG. 14, and the rates of increase/decrease in secondary mutation signals at the time of primary mutation diagnosis (t1) and the time of secondary mutation diagnosis (t2) are shown in FIG. 15. Especially, signals determined as primary mutation were visualized with yellow boxes, and signals determined as secondary mutation were visualized with purple boxes.

As a result of performing treatment monitoring on patients, the number of proteins with secondary mutations increased depending on the time of diagnosis, and thus the results suggested that the present invention can be applied as a novel mutation screening method.

### EXPLANATION OF REFERENCE NUMERALS

100: computing device
200: SERS signals of exosomal proteins
300: neural network model
310: convolutional neural network
320: fully-connected neural network
T₁: first transformation
T₂: second transformation
S100: input data generation step
S200: mutated exosomal protein detection step
S300: mutated exosomal protein classification step

### INDUSTRIAL APPLICABILITY

The present invention relates to a method and an apparatus capable of detecting and monitoring the mutations of exosomal proteins by classifying surface-enhanced Raman scattering (SERS) signals of proteins contained in exosomes, which are abundant in the blood and stably transport a biomarker, through a convolutional neural network (CNN)-based artificial intelligence algorithm, and mathematically analyzing the derived classification results, wherein the method and device according to the present invention can overcome the limitations of signal heterogeneity that are inherent in existing techniques, and thus enable convenient and prompt non-invasive liquid biopsy on exosomal proteins in the plasm.

1. A method for analyzing mutations of exosomal proteins in a specimen, the method being executed by a computing device, the method comprising:
a generation step of generating input data based on surface-enhanced Raman scattering (SERS) signal data of the exosomal proteins in the specimen; and
a detection step of determining the mutation status of the exosomal proteins on the basis of the input data, by using a pre-trained detection model.

2. The method of claim 1, comprising a signal measurement step of measuring SERS signals from the exosomal proteins in the specimen.

3. The method of claim 1, wherein the generation step comprises a statistical processing step of transforming multiple SERS signal data points into the mean signal through statistical processing.

4. The method of claim 3, wherein in the statistical processing step, 80 to 100 SERS signal data points are transformed into the mean signal through statistical processing.

5. The method of claim 1, wherein the detection step comprises:
a production step of producing output information on the mutation status of the exosomal proteins by using a pre-trained detection model; and
a similarity determination step of determining the relative similarity of the exosomal proteins in the specimen with wild-type and mutated exosomal proteins, on the basis of the output information.

6. The method of claim 5, wherein the output information is one-dimensional tensor data with a length of 128.

7. The method of claim 1, wherein the detection model is trained through supervised learning on the basis of: training data containing SERS signal data generated from wild-type exosomal proteins and SERS signal data generated from mutated exosomal proteins; and
detection labels with respect to the mutation status of the exosomal proteins corresponding to the training data, and
the supervised learning is performed using the detection model, on the basis of: training information generated from the training data by using the detection model; and the detection labels.

8. The method of claim 1, wherein the method comprises a classification step of, when the exosomal proteins are determined as being mutated in the detection step, classifying the mutation order of the mutated exosomal proteins on the basis of the input data by using a pre-trained classification model.

9. The method of claim 8, wherein when the exosomal proteins are determined as being mutated in the detection step, the classification step is performed on exosomal proteins in a specimen that is newly obtained from the same subject after the lapse of a predetermined period following the termination of the detection step.

10. The method of claim 8, wherein the classification step comprises a mutation information generation step of generating mutation information by calculating the proportion of SERS signal data points determined as showing secondary or higher mutations among the total SERS signal data points.

11. The method of claim 10, wherein the total SERS signal data points are generated on the basis of 100 SERS signals measured from the exosomal proteins in the specimen.

12. The method of claim 8, wherein the classification model is trained through supervised learning on the basis of: order training data containing SERS signal data according to the mutation order of the exosomal proteins; and classification labels with respect to the mutation order of the exosomal proteins corresponding to the order training data, and
the supervised learning is performed using the classification model, on the basis of: training information generated from the order training data by using the classification model; and the classification labels.

13. A computer program stored in a storage medium, wherein, when the computer program is executed on at least one processors, the computer program performs operations for analyzing mutations of exosomal proteins in a specimen, the operations including:
an operation of generating input data based on SERS signal data of exosomal proteins in a specimen; and
an operation of determining the mutation status of the exosomal proteins on the basis the input data by using a pre-trained detection model.

14. A computing device for analyzing mutations of exosomal proteins in a specimen, the computing device comprising:
a processor comprising at least one core; and
a memory,
wherein the processor generates input data based on SERS signal data of exosomal proteins in a specimen, and
determines the mutation status of the exosomal proteins on the basis the input data by using a pre-trained detection model.
